(19) United States
(12) Patent Application Publication
(10) Pub. No.: US 2023/0238140 A1
(43) Pub. Date: Jul. 27, 2023

(54) ADDICTION TREATMENT AND MANAGEMENT

(71) Applicant: David Reeser, Wilmington, NC (US)

(72) Inventor: David Reeser, Wilmington, NC (US)

(21) Appl. No.: 17/584,460

(22) Filed: Jan. 26, 2022

(51) Int. Cl. G16H 50/30; G16H 70/60

(52) U.S. Cl. CPC G16H 50/30; G16H 70/60

(57) ABSTRACT

A system and method of managing addiction recovery may include importing all-source medical records for patients, as well as generating and storing models based on the medical records. The system may receive real-time client event data, not limited to biometric, clinical, locational, and behavioral data. The system and associated processes may generate and update client model based on real-time client event data. The client model may be compared to the stored model to assess treatment progress and risk. Where appropriate, a caregiver may be immediately alerted to facilitate telemedical and other immediate intervention. The system may present the provider with a visual representation of assessment (e.g., based in part on established indexes and treatment metrics), along with predictive analysis and prescription verification. Provider notes may be seamlessly uploaded and coordinated with billing to continuously update the medical records. The system may additionally breakout and store clinic and patient demographics, as well as success rates for different treatment scenarios and locations.

### BACKGROUND

Opioid addiction is an epidemic. The only treatment scientifically shown to consistently improve recovery from opioid use disorder (OUD) is medically assisted treatment (MAT). In order for this; treatment to have long term success, patients need to remain in care for 12-18 months. Unfortunately, two-thirds of patients who begin treatment quit in less than six months. Increasing retention in MAT programs is key to improving outcomes and reducing the cost of treating OUD and other forms of substance use disorder (SUD).

Unfortunately, MAT centers clinics may lack an ability to analyze their own data. Conversely, data science companies can lack subject matter expertise with MAT data.

### SUMMARY

A system and method of managing addiction recovery may include importing all-source medical records for patients, as well as generating and storing models based on the medical records. The system may receive real-time client event data, not limited to biometric, clinical, locational, and behavioral data. The system and associated processes may generate and update client model based on real-time client event data. The client model may be compared to the stores model to assess treatment progress and risk. Where appropriate, a caregiver may be immediately alerted to facilitate telemedical and other immediate intervention.

The system may present the provider with a visual representation of assessment (e.g., based in part on established indexes and treatment metrics), along with predictive analysis and prescription verification. Provider notes may be seamlessly uploaded and coordinated with billing to continuously update the medical records. The system may additionally breakout acid store clinic and patient demographics, as well as success rates for different treatment scenarios and locations.

A particular embodiment of a system may include an algorithm for the detection of acute use and quantification of withdrawal via a wearable device. The system may be supported by cloud based technologies. A daily withdrawal curve may be generated and presented. The presented information may include a specific resiliency score for patients diagnosed with substance use disorder/opioid use disorder.

The system additionally may include trending and predictive forecasting of behavioral metrics based on machine learning. An alerting feature may notify clinicians when thresholds have been Exceeded, prompting human interaction with interface and medically appropriate patient interactions. For instance, a patient may miss an appointment and be assigned a new depression score that is two standard deviations from normal. In this example, the system may alert clinicians and/or provide a button that won't let a provider proceed in software until they select a next step on the screen to contact the patient, among other actions.

A particular embodiment may use biometrics to distinguish between physiologic stress and psychological stress in order to aid clinical treatment design, as well as dosage considerations in MAT. Telehealth integration with decision support and biometric insights may be automatically generated and displayed.

In a sense, the system may provide an agnostic pipeline to normalize and transform data for biometric insight displayed in software. The system may provide relapse risk stratification for clinical decision support. This support may be intended for clinicians that are well versed in SUD treatment, as well as those with only an x-waiver (e.g., eight hours of training in MAT) to triage more effectively and provide just in time interventions when appropriate.

The system may comprise an EHR decision support tool designed for medically assisted treatment, to bolt on to the clinic's EHR and integrate directly into the health information exchange (HIE). In this manner, the system may create a patient care pathway for larger community studies. Generated data may comprise study pathways that lead to relapse, as well as those that lead to recovery in order to improve the machine learning algorithms of the platform.

A particular embodiment may include push button launch/installation of a decision support tool for opioid and substance use disorder (e.g., Kareo direct linkage). The system may provide patient mapping and geographical analysis for supporting operational decision making. An application of the s/stem may allow for sending patient facing dashboard, assessments, and data collection with white label capabilities for the clinic/medical practice.

Techniques described herein enable automated CPT coding, artificial intelligence based recommendations for substance use disorder/opioid use disorder. Mother or the same embodiment may provide patient interaction and metrics driven PDF export for patient billing support. Natural language processing (NLP) of clinical notes may be supported in order to feed resiliency and relapse predictive artificial intelligence models. The system may include business intelligence capabilities to generate reports on patient build with every searchable field to improve resource allocation and operational decision making,

The above description is provided as an overview of only some implementations disclosed herein. Those and other implementations are described in more detail here. In addition, some implementations include one or more processors of one or more computing devices, where the one or more processors are operable to execute instructions stored in associated memory, and where the instructions are configured to cause performance of any of the methods described herein. Some implementations also include one or more non-transitory computer readable storage media storing computer instructions executable by one or more processors to perform any of the methods described herein.

It should be appreciated that all combinations of the foregoing concepts and additional concepts described in greater detail herein are contemplated as being part of the subject matter disclosed herein. For example, all combinations of claimed subject matter appearing at the end of this disclosure are contemplated as being part of the subject matter disclosed herein.

### BRIEF DESCRIPTION OF THE DRAWINGS

FIG. 1 is a block diagram of an embodiment of a system that includes program code that imports electronic health record (EHR) and biometric data to improve outcomes for substance use disorder (SUD) patients;

FIG. 2 is a block diagram of an implementation of a system configured to manage addiction recovery by importing all-source medical records for patients, as well as generating and storing models based on the medical records and real-time client event data, not limited to biometric, clinical, locational, and behavioral data;

FIG. 3 shows a graph of biometric data (i.e., a heart rate) that is used by software of an embodiment to determine events that serve as inputs to algorithms that recommend courses of treatment for patients;

FIG. 4 shows a screenshot of a display of an embodiment of a system;

FIG. 5 shows a screenshot of a display of an embodiment of a system that may be generated by a user clicking on the patient specific profile, such as may be linked to by a button of FIG. 4;

FIG. 6 shows a screenshot of a display of an embodiment of a system that includes an example of the condensed report that is generated from a decision tool specific to the selected patient folder;

FIG. 7 shows a screenshot of a display of an embodiment of a system that includes an example of subcategories presented by software that may allow a medical professional to visualize patients across one or many locations, according to status, diagnosis, behavior scores, insurance providers, demographics, etc.;

FIG. 8 shows a screenshot of a display of an embodiment of a system that includes an example of a user tab allowing an administrator to add or delete users within the system, or to change their level of access;

FIG. 9 is a flowchart of an embodiment of a method that may be executed by an embodiment of a system described herein to retrieve and real-time data from multiple sources, and to analyze the data to inform care providers prior to updating patient records;

FIG. 10 is a flowchart of an embodiment of a method that may be executed by an embodiment of a system described herein to grant access to and about care providers, as well as to update records of clinician performance;

FIG. 11 is another block diagram of an example environment in which implementations disclosed herein may be implemented; and

FIG. 12 illustrates an example architecture of a computing device.

### DETAILED DESCRIPTION

A system and method of managing addiction recovery may include importing all-source medical records for patients, as well as generating and storing models based on the medical records. The system may receive real-time client event data, not limited to biometric, clinical, locational, and behavioral data. The system and associated processes may generate and update client model based on real-time client event data. The client model may be compared to the stored model to assess treatment progress and risk. Where appropriate, a caregiver may be immediately alerted to facilitate telemedical and other immediate intervention. The system may present the provider with a visual representation of assessment (e.g., based in part on established indexes and treatment metrics), along with predictive analysis and prescription verification. Provider notes may be seamlessly uploaded and coordinated with billing to continuously update the medical records. The system may additionally breakout and store clinic and patient demographics, as well as success rates for different treatment scenarios and locations.

An embodiment of the system may include program code that imports electronic health record (EHR) data to improve outcomes for substance use disorder (SUD) patients. Inputs to system algorithms may include biometric data.

An embodiment of the system may include a mobile physiological monitoring system that includes wearable devices. Opioid use may be determined by a comparison with a baseline value. Pattern analysis may be used, and machine learning algorithms may be employed to detect specific psychological states within an individual. An embodiment of the system may explore a key period from the baseline measurements prior to initiation of a drug. For instance, a thirty thy period may be monitored. The system may objectively measure a patient's self-administration and physiological response to opioids at each administration event. An embodiment of the system may model changes and opioid response with the individuals. The system may compare responses between individuals.

Measuring proximal risk may include a user wearing a wristband or other wearable device. The wristband may be attached using hook and loop fasteners or a wristband, for example. The wristband or other wearable device may collect cardiovascular activity, electrodermal activity, and skin temperature data, as well as motion-based activity in the anterior posterior medial and lateral positions, in addition to motion along a z-axis. The system may be continuously updated at a predetermined sampling rate. An illustrative sampling rate may comprise eight cycles per second.

A cloud based software system may generate data driven actionable insight to improve addiction recovery. An embodiment of the system may enable medication assisted treatment (MAT) centers to acquire data from real patients to build, test, and iterate evidence based processes.

The system may leverage three types of data to improve patient retention in clinics: human behavioral data, electronic medical record (EMR) data, and biometric data. The system may bring clinical, governmental (e.g., law enforcement), data science, cloud, and data security expertise to the MAT environment.

An embodiment of the system may provide a web-based dashboard providing data driven insight to assist clinicians in decision making. This may be accomplished by converging behavioral data and EHR in addition to biometric data from devices worn or used by patients (e.g., a phone, patch, or anklet). An embodiment of the system may comprise technology assisted care (TAC) and may improve patient retention in MAT programs.

A particular embodiment of the system may address opioid addiction treatment in a manner that it is built on massive amounts of procured data collected from all the public and private organizations addressing this epidemic (e.g., rehabilitation clinics, hospitals, police and other emergency personnel, civic, faith based organizations and local government organizations). That delta may then be automatically analyzed by cloud servers to determine patterns the human mind is incapable of finding because of the size and complexity of the data sets. The patterns may reveal correlations that are directly related to the success of the rehabilitation so that professionals working with recovering individuals may be able to have better and more actionable information to help them.

The system may generate a community based communication tool providing each organization an ability to interact with a platform of an embodiment of the system in a manner that may increase the effectiveness of their efforts, improve communications among other partners, and reduce c went redundancies in spending and time consuming activities.

The system may incorporate wearable technology utilizing inherent biometric capabilities to detect cravings that when a treated individual may use or recently abused drugs. Detecting these findings are made possible by filtering biometric data with algorithms that reveal these occurrences as signatures in near real-time. The signatures may be used to alert either emergency personnel, family, or rehabilitation specialists about the event. Input to the algorithm may include whether an addict is geographically detected or known to be near a location known for drug dealing and use. An embodiment of the system may include a wearable adapted to d liver a drug to reverse an overdose, once detected.

An embodiment of the system uses a cloud based technology platform to address two challenges facing the fight against opioid addiction: a coordinated, data-driven approach to rehabilitation and communication, and a more strategic way to monitor those undergoing outpatient treatment for opioid addiction. The system may collect data from which machine learning may be performed. Program code may apply the machine learning to generate custom treatment plans for an addict. As discussed herein, data may be linked from a wearable device to a platform of the system to monitor the progress of a person's recovery.

The data co lection, machine learning, and treatment plan generation may be independent of the wearable-related information. The system may generate a custom treatment plan in minutes, as opposed to months. This generation feature may result in a significant reduction in the cost of rehabilitation. The feature may additionally result in less intervention by support staff (e.g., medical personnel, rehabilitation professionals, and police). The system may ultimately result in fewer deaths, relapses, and medical complications.

The wearable device may provide a predictive capability that may recommend prescriptions. Wearable health technology may include program code to measure and collect data that has the capacity to detect unique patterns that could identify someone has used or is about to use; especially if the person is in an area where they have gotten their supply in the Past.

An embodiment of the system may enable tiered access and pricing depending on a priority or need of a requesting entity. For instance, healthcare clinics may need a significant number of features within the platform, while civic or faith based groups may only utilize a small portion of the features.

Additional embodiments may include a clinic portal to enable clinicians to access real-time patient data. Another or the same embodiment may include score trending and use artificial intelligence to perform predictive analysis. The predictive artificial intelligence may monitor all trends to anticipate potential trends and trouble spots. An embodiment may enable patient interaction to include accesses a patient interaction report, as well as to view notes and send notes.

According to another aspect, the system may include resiliency scoring. Resiliency scoring may include a metric score or visual representation of a patient's progress in treatment and potential for relapse. An example of a system may include an interface with EHR integration. The integration may provide additional integration of data used as decision inputs to the algorithms. The system may import biometric insights as well as clinical notes. For example, the system may monitor heart rates and temperatures, as well as process.

Another embodiment may apply artificial intelligence algorithms to perform Current Procedural Terminology (CPT) coding recommendations. For instance, the algorithms may receive reporting data and automatically categorize it according to the latest CPT codes.

Additional embodiments may integrate telehealth capabilities. Kareo direct linkage may be made incorporated and facilitated by the program code, as well as EPIC software Integration.

According to another particular embodiment, the system may include patient mapping and geographic analysis. Embodiments of the system may include patient facing assessments tied to authentication and documentation. Artificial intelligence may be used to find trends, such as with trends associated with patient withdrawal.

While certain embodiments lend themselves particularly to addiction treatment, one skilled in the art w II appreciate that the techniques and hardware may have application in other medical monitoring endeavors, including monitoring viral and bacterial outbreaks, as well as chronic disease treatment and mental illness care.

An embodiment of the system may include a biometric algorithm that may detect acute use and quantify ng levels of withdrawal of individuals in treatment for substance use disorder with the use of wrist worn devices. For example, the system may collect 40,000 seconds of data. The system may normalize frequencies of sensor data to one second intervals with specific focus on three-point accelerometry, electrodermal activity (EDA), heart rate variability (HRV), heart rate (HR), interbeat interval (IBI), skin temperature and saturation of peripheral oxygen (SpO₂).

An embodiment of the system may use machine learning to enable supervised and unsupervised data. The system may normalize the data to one second intervals and events may be labeled to Indicate acute use. The escalation or decline of physiological parameters may be input to algorithms to identify withdrawal symptoms. Program code may interpret biometric waveforms and output results such that physicians treating patients with substance use disorder are able to personalize the treatment approach.

Techniques may have particular utility in improving retention in the induction, or beginning of MAT, when patients are the most prone to overdose and treatment failure. Data may be displayed and automatic recommendations may be output to provide insight into this early time of treatment dosing and timing of the appropriate MAT medication (e.g., buprenorphine, methadone, etc.). The medication may be adjusted to an appropriate level to adequately treat the patient according to the recommendation. In this manner, the system may simultaneously decrease the unhealthy drug seeking behavior of those that are actually intending to abuse the medications.

A particular embodiment may be device and software agnostic in order to leverage the currently available hardware already familiar and accessible to patients. The system may feed back the interpreted data into a decision support tool within the specific patient profile. The biometric data Mal be displayed as a daily withdrawal curve indicating the escalation or decline of withdrawal throughout the day and over an extended period of time.

Acute events of outside use of prescribed medications may also be automatically registered as an event within the folder. This data may be used as input to the algorithms to tailor treatment approaches that include counseling, dosing protocol and lab testing. Biometric insights may be used b f the system to accomplish monitoring and chronic disease management. Passive data collection may be used to automatically personalize treatment approaches and provide objective insight into individual recovery. Additionally, the algorithm may save battery consumption by adjusting rapid measurements to take place around events as identified to machine learning to make the most efficient use of the hardware. Key window of time measurement may include one hour before MAT dosing and 40 minutes after a dose has been administered.

The system may use biometric insights and biomarkers to assist in tapering patients off opiates. Algorithm; may identify the progress and trajectory of recovery and display results to the physician, patient, and anyone within the community of recovery of that patient. The system may use biometric specific algorithms to help patients safely switch from one opiate to another prescribed one. Medication changes may prompt the system to monitor individuals undergoing a medication switch. Program code may quantify and separate physical withdrawal and psychological withdrawal. In one example, a patient being treated for chronic pain may particularly benefit, whether or not they have a pre-existing diagnosis of substance use disorder.

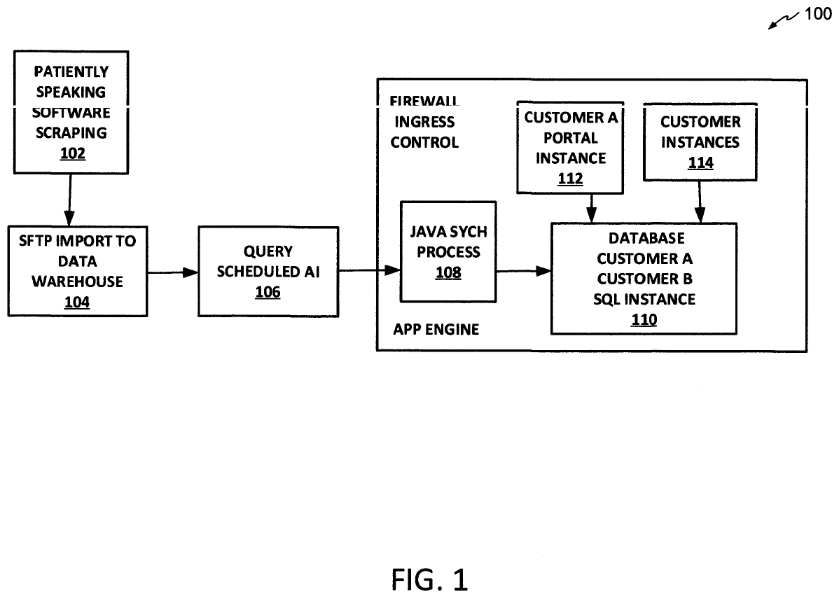

FIG. 1 is a block diagram of an embodiment of a system that includes program code that imports EHR data to improve outcomes for substance use disorder (SUD) patients. Inputs to system algorithms may include biometric data. The architecture described in FIG. 1 operates within cloud-based environment to integrate electronic health records.

An embodiment of the system comprises a decision support tool that bolts onto an existing EHR data to extract data that is already available. The system presents the data in a clear and actionable format. Machine learning may be applied to aggregated data to stratify the risk of individual pi dents that are trending toward a relapse or not meeting their clinical care plan. Each patient may also be assigned a resiliency score that indicates their risk of relapse, along with predictive scores of ongoing levels of depression, anxiety, sleep quality and other behavioral metrics that can add depth and texture to decision making in this very sensitive population whose success rate is very low.

Turning moue particularly to FIG. 1, a patiently speaking medical software module 102 (e.g., Kareo) scraping module may include bots that are programmed to scrape specific data from the medical software online.

An SFTP Import to a data warehouse (e.g., BigQuery) module 104 may deposit data from the hots into a Secure File Transfer Protocol (SFTP) server. The module 104 may further link to an API and send a push notification to the cloud initiating a data pull. Alternatively, the module 104 may perform a scheduled data pull of the new data scraped from medical software (e.g., every 24 hours from Kareo).

A scheduled artificial intelligence (AI) module 106 may aggregate and transform specific formats to populate predefined tables in databases inside a cloud platform.

A Java sync process module 108 may receive the output databases from the scheduled AI module 106 to populate the client facing platform in decision support tool inside of a MySQL instance 110.

The MySQL instance module 110 may be updated with incoming data. Such data may include biometrics, labs, clinical notes, and appointment data, among other types of data. Each customer's (e.g., clinic's) patient data may be separated so that only data specific to that group is stored and accessible inside the software platform.

A customer portal instance module 112 may comprise a unique (e.g., for each user), permission based access to the appropriate data on the patients in their care, as separated by organization. Connection to the patient database may be facilitated by an SSL secure connection inside of a virtual private cloud (VPC).

A customer Instances module 114 may comprise a permission based connection with the same SSL connection to the database, which allows the user to add notes and other data when interacting with the software. Clinician's actions are then stored in that patients record for continuity of care, billing and compliance purposes.

The system 100 may assist providers who treat patients in the SUD field, as well as primary care physicians with very little training in SUD and behavioral medicine. For primary care professionals, an embodiment of the system may be particularly useful as it guides their attention to gold standard protocols. The recommendations and outputs of the system 100 may allow such provider; to be more effective at decision making with a type of chronic disease in patients that they do not frequently treat.

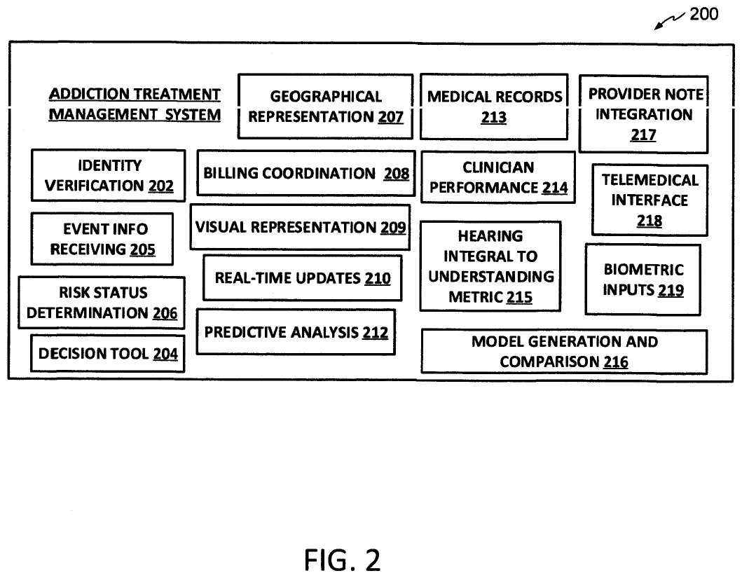

FIG. 2 is a block diagram of an implementation of a system configured to manage addiction recovery by importing all-source medical records for patients, as well as generating and storing models based on the medical records and real-time client event data, not limited to biometric, clinical, locational, and behavioral data. The illustrative system 200 of FIG. 2 may be similar to the treatment management module 102 of FIG. 1.

The implementation of the system 200 includes multiple modules 202, 204, 205, 206, 207, 208, 209, 210, 211, 212, 213, 214, 215, 216, 217, 218, and 219 to automatically manage and facilitate addiction treatment. For example, risk status determination module 206 may be included in the system 200. The risk status determination module 206 may be configured to assign a status to a patient based on their susceptibility to relapse or otherwise their stage of recovery.

A decision tool module 204 may include algorithms to automatically assess risks and to initiate treatment plans of action. A geographical representation 207 may enable the system to show maps and other geographic graphics of where clinics and individual or populations of patients are located.

A billing coordination module 208 may be configured to automatically generate bills based on input provider interaction. For instance, a call may be logged by the system and converted to an invoice with a billing code. The system 200 may additionally include a visual representation module 209 configured to display patient data in a manner that is readily apparent to providers. For instance, the visual representation module 209 may display graphs that include slopes and peaks indicating events or trends.

The system 200 may additionally may include a real-time updates module 210. The real-time updates module 210 may provide continuous feedback and inputs for timely updates. For example, biometric and location data may be monitored and automatically provided to algorithms as inputs. A predictive analysis module 212 may use artificial intelligence and modeling to anticipate trends and events.

A medical records module 213 may be included in the system 200. The medical records module 213 may be used to import and populate data fields used in algorithms of some embodiments. A clinician performance module 214 may be used to record and evaluate results of different clinics and other providers.

Other modules included in the system 200 may include a hearing integral to understanding a metric module 215, as well as a model generation and comparison model 216. A provider notation integration module 217 may prompt, organize, and assimilate clinician notes for sharing and inputting into algorithms. The system 200 may additionally include a telemedical interface 218, as well as a biometrics inputs module 219 to receive, store, and process biometric data as algorithmic input.

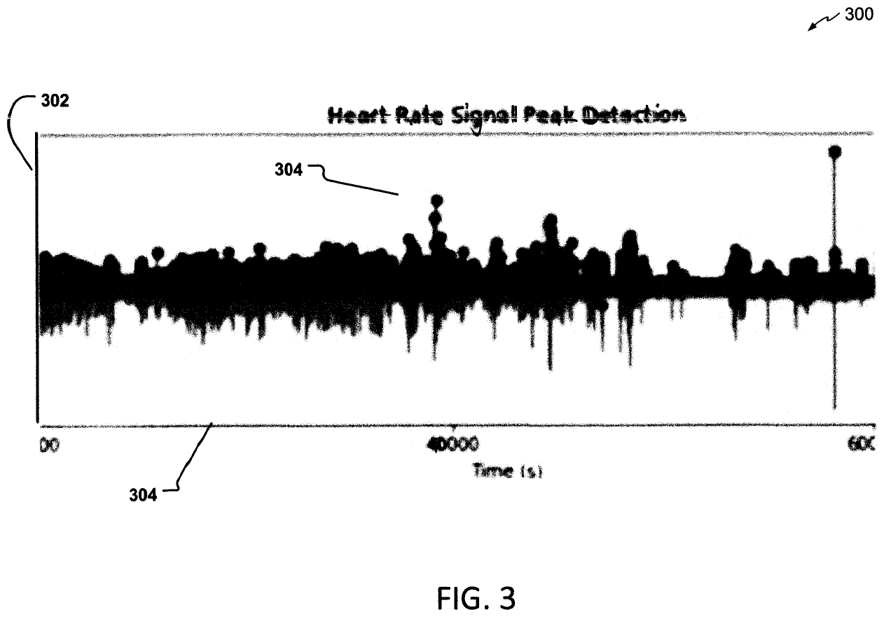

FIG. 3 shows a graph 300 of biometric data (i.e., a heart rate) that is used by software of an embodiment to determine events that serve as inputs to algorithms that recommend courses of treatment for patients. The graph includes a plot of heart beats per minute on the y-axis 302 versus time on the x-axis 304. According to one embodiment, a watch worn by a patient may sense the heartrate. The wearable device may additionally provide a predictive capability. For instance, software may use the heartbeat information to recommend prescriptions. Wearable health technology may include program code to measure and collect data that has the capacity to detect unique patterns that could identify someone has consumed medications or illicit drugs or is about to; especially if the person is in an area where they have procured or consumed these drugs in the past. For example, a peak 306 in the heartrate may coincide with a stressful and vulnerable moment for a patient when this data is combined with other data collected from sensors and behavioral assessments, etc.

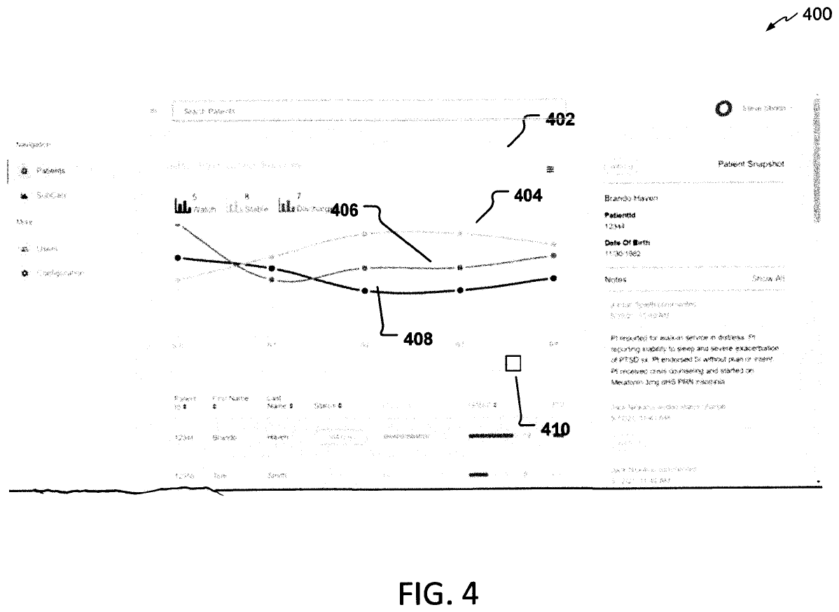

FIG. 4 shows a screenshot 400 of a display of an embodiment of a system. The display 400 may be on a cellular phone, for instance, or on a device similar to the display of the device shown in FIG. 1. Once logged in, a clinician may be able to see an aggregated list of their patients being treated for SUD that are in their care. Psychometric scores and other relevant data specific to the treatment of SUD may be presented in a simple and logical format. The display 400 may save time while presenting the most relevant data needed by a physician to make a data informed decision. Such decisions may involve emergency interventions to prevent relapse, as well as triage to a patient population to better understand who needs more attention based on who has greatest need that day.

A dashboard 402 of the screenshot 400 shows all the current psychometric scores of the patients. First, second and third lines 404, 406, and 408 indicate three distinct statuses, or buckets, of patients.

The first line 404 (e.g., stable status) represents patients that are maintaining the predicted and prescribes care plan. The second line 406 (discharged status) includes patients that have completed care and have been released from care, or left against medical advice. The third line 408 (e.g., watch status) represents those patients that are currently in care and are thought to pose some risk of relapse for at least one or more reasons.

One such reason may include a clinician having marked a patient as watch status may be due to the patient displaying behavior and physical signs that indicate that they are unstable. Another such reason may include the patient having completed a psychometric assessment that has exceeded an acceptable threshold generating an alert in the system to the physician and becoming a watch status. Still another example of a reason may include one or more appointments have been missed in a row, along with many other supporting factors built into our machine teaming algorithm that considers social, demographic, other health conditions, mental heals h diagnoses and labs to determine if the patient is meeting the treatment prescribed. A selectable box 410 may be a patient specific file for additional details, including clinical notes.

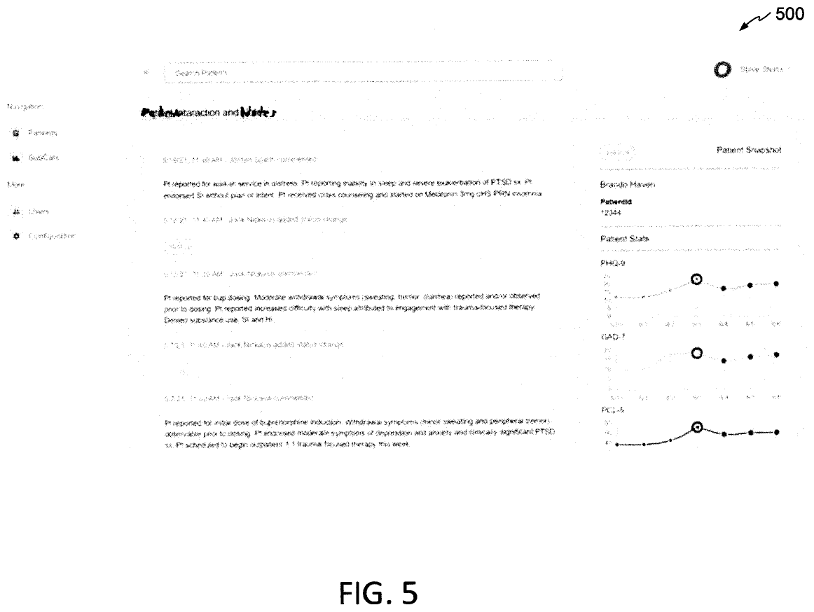

FIG. 5 shows s screenshot 500 of a display of an embodiment of a system that may be generated by a user clicking on the patient specific profile, such as may be linked to by the button 410 of FIG. 4. The display shows succinct clinical interactions over time on a single patient that has been marked as watch, and therefore at a higher potential risk of relapse. The display presents relevant notes and status changes over time for the clinician to easily see when the patient began to decline based on the subjective notes of other care providers.

These notes may then be updated by clinically valid psychometric scores that indicate that the patient: has exceeded the acceptable range supporting the observation of the treating clinician. These assessment scores are also trended over time in order to show the trajectory of the patient. The circles indicate a current score, while dots that follow are predictions of what the score may be when the assessment is given again. These predictive behavioral metrics are derived through machine learning on aggregate data sets that look at patterns in behavior that lead to relapse and successful treatment as well, and then doing a regression analysis to plot how the behavioral scores for the individual assessments changed based on that outcome. In this way, the projected behavioral metrics act like a prediction of the weather in the future, the moving of a cold front.

Features may enable a clinician or other medical professional to download a report of the data driven findings presented on the screen to support billing, or to share with other clinicians outside of the clink (e.g., should the patient be released for another level of patient care).

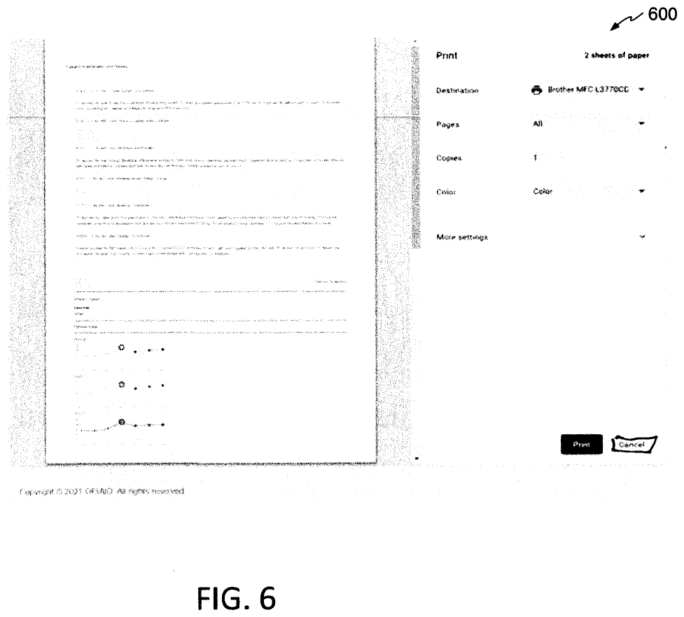

FIG. 6 shows a screenshot 600 of a display of an embodiment of a system that includes an example of the condensed report that is generated from a decision tool specific to the selected patient folder. The report may be printed or exported securely.

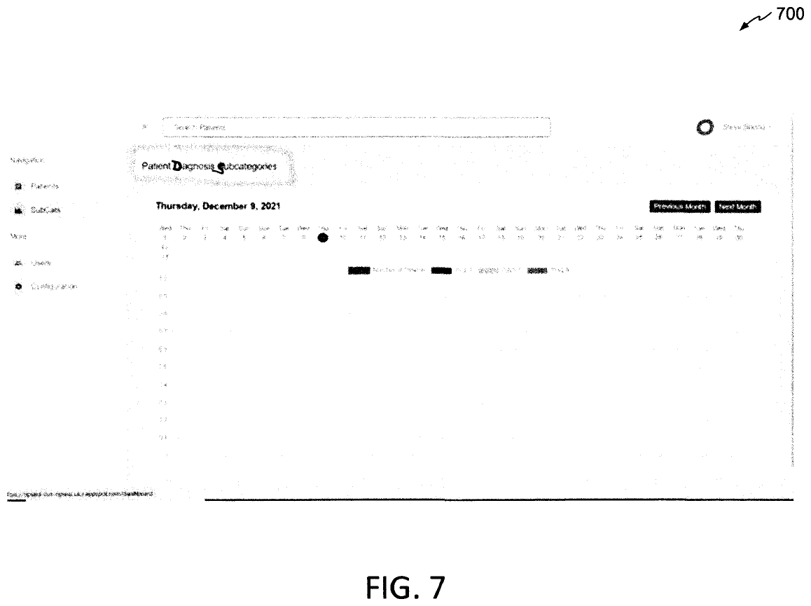

FIG. 7 shows a screenshot 700 of a display of an embodiment of a system that includes an example of subcategories. Subcategories presented by software allows the medical professional to visualize patients across one or many locations, according to status, diagnosis, behavior scores, insurance providers, demographics, etc. This feature may provide operational decision making that can impact resource allocation of staff and medication to decrease burnout, improve patient flow and ultimately lead to improved patient outcomes due to efficiencies created by data driven operational decision making.

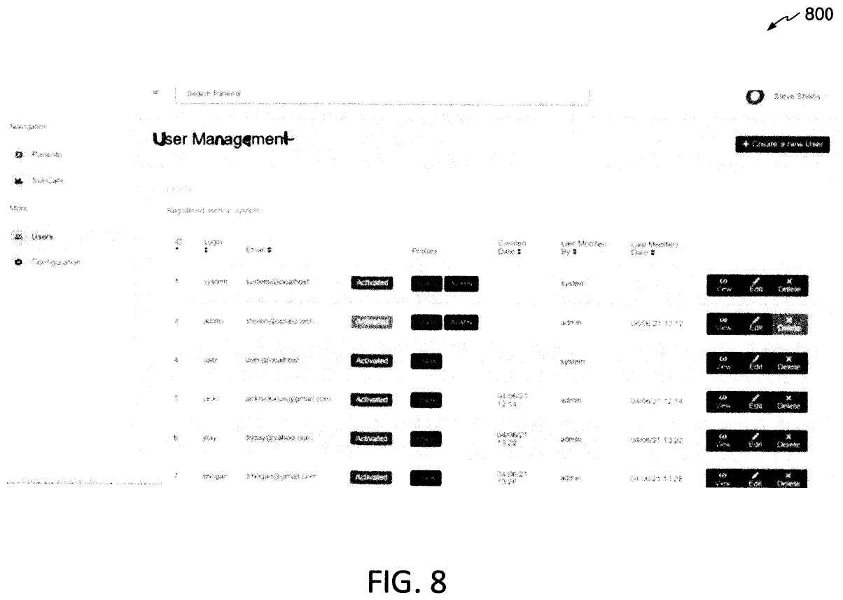

FIG. 8 shows a screenshot 800 of a display of an embodiment of a system that includes an example of a user tab allowing an administrator to add or delete users within the system, or to change their level of access. The system may additionally create an audit trail in case user history is nee led upon an accreditation evaluation or inspection for compliance. This feature may help to promote compliance for data security.

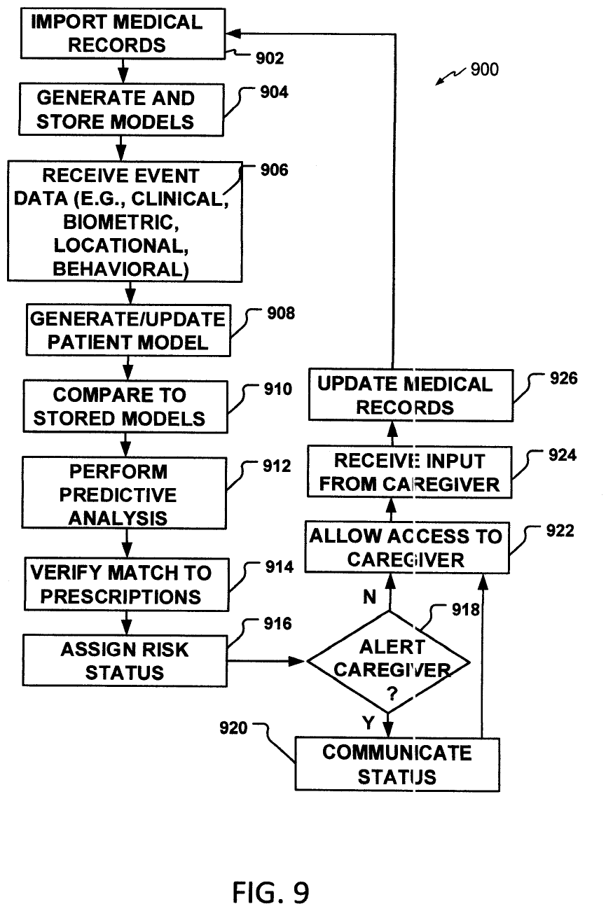

FIG. 9 is a flowchart of an embodiment of a method 900 that may be executed by an embodiment of a system described herein to retrieve and real-time data from multiple sources, and to analyze the data to inform care providers prior to updating patient records. Turning more particularly to the flowchart, the method 900 may include importing medical records at 902.

The system may generate and store models at 904, and receive event data at 906. Examples of event data may include clinical, biometric, locational, and behavioral event data.

According to a particular embodiment, the method 900 may include update or generating new patient models at 908. At 910, the system may compare the generated models to stored models.

The system may perform predictive analysis at 912. The system at 914 may verify match to prescriptions, as assign a risk status at 916. They system at 918 may determine if the assigned risk status warrants alerting a caregiver. When warranted at 918, the system may communicate the status to a professional provider at 920.

In any case, doctors and other caregivers may access analysis and data at 922. The caregiver at 924 may choose to provide input into the system. Any input from the caregiver may be use at 926 to update patient medical records. The method 900 may continuously update records and patient statuses.

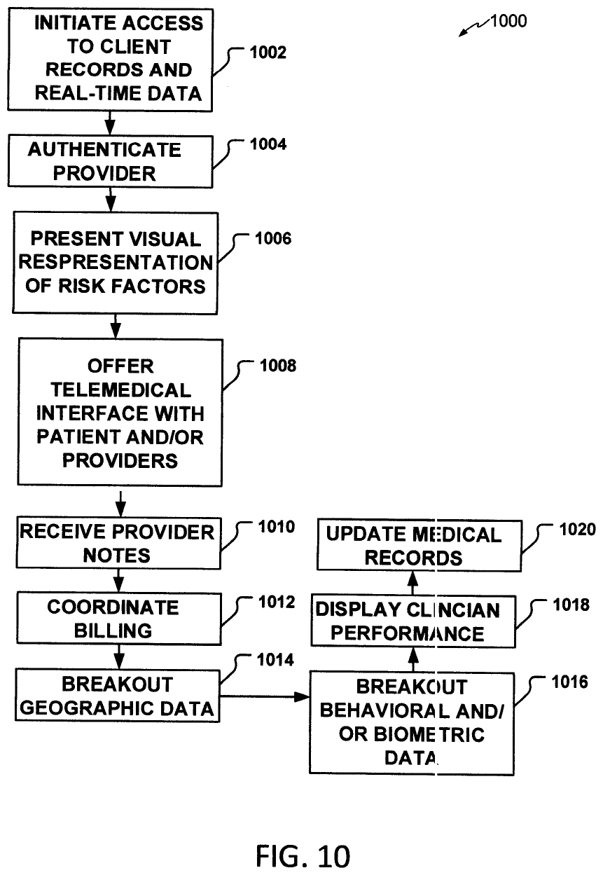

FIG. 10 is a flowchart of an embodiment of a method 1000 that may be executed by an embodiment of a system described herein to grant access to and about care providers, as well as to update records of clinician performance. Turning more particularly to the flowchart, the method 1000 may initiate access to client records and real-time data at 1002.

At 1004, the system may authenticate a provider. After authentication, the system at 1006 may present a visual representation of risk factors. For instance, the system may generate graphs for a provider to readily view and digest.

A telemedical interface option may be provided at 1008 for either or both patients and providers. The system 1010 may receive provider notes, and billing at 1012 may be automatically coordinated with provider activity.

The system may at 1014 may breakout geographic data, as well as searchable behavioral and biometric data at 1016. Clinician performance data may be searched and displayed at 1018, and medical records may be updated at 1020.

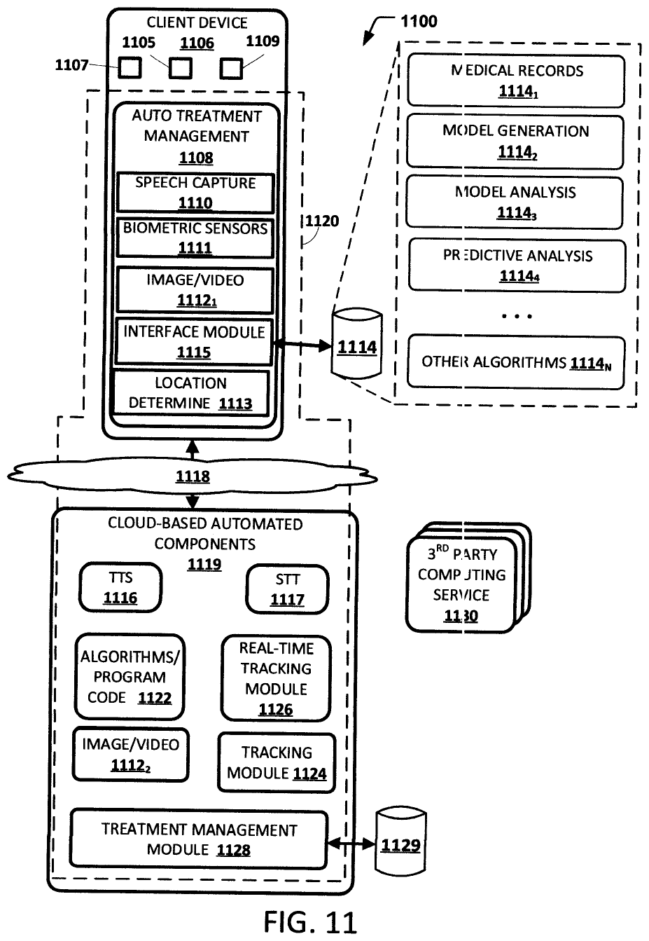

Now turning to FIG. 11, an example environment, apparatus, or system 1100 in which techniques disclosed herein may be implemented is illustrated. The example environment includes one or more client computing devices 1106. Each client device 1106 may execute a respective instance of an automated treatment management client 1108, which may also be referred to herein as a client portion of a system 1100. One or more cloud-based automated components 1119, which may also be referred to herein collectively as a server portion of a treatment management system 1100, may be implemented on one or more computing systems (collectively referred to as a cloud computing system) that are communicatively coupled to client devices 1106 via one or more local and/or wide area networks (e.g., the Internet) indicated generally at 1118.

In various implementations, an instance of an automated client 1108, by way of its interactions with one or more cloud-based automated components 1119, may form what appears to be, from the user's perspective, a logical instance of spoken, written, sensor generated, machine language or other data communication 1120 with which the user may engage or be engaged by the system. One instance of such an automated data exchange module 1120 is depicted in FIG. 11 in dashed line. It thus should be understood that each user that engages with an automated client 1108 executing on a client device 1106 may, in effect, engage with his or her own logical instance of system exchange module 1120. For the sake of brevity and simplicity, the terms, system and apparatus, as used herein as serving a particular user will refer to the combination of an automated client 1108 executing on a client device 1106 operated by the user and one or more cloud-based automated components 119, which may be shared amongst multiple automated clients 1108. It should also be understood that in some implementations, the system 1100 may interact with any user regardless of whether the user is actually served by that particular instance of a data exchange module 1120.

The one or more client devices 1106 may include, for example, one or more of: a desktop computing device, a laptop computing device, a tablet computing device, a mobile phone computing device, a computing device of a vehicle of the user (e.g., an in-vehicle communications system, an in-vehicle entertainment system, an in-vehicle navigation system), a standalone interactive speaker (which in some cases may include a vision sensor), a smart appliance such as a smart television (or a standard television equipped with a networked dongle with automated capabilities), scanner, and/or a wearable apparatus of the user that includes a computing device (e.g., a watch of the user having a computing device, glasses of the user having a computing device, a virtual or augmented reality computing device). Additional and/or alternative client computing devices may be provided. Some client devices 1106, such as standalone interactive speakers may take the form of devices that are primarily designed to facilitate dialog between users and data exchange modules. Some such devices may take the form of a standalone interactive speaker with an attached display, which may or may not be a touchscreen display.

In some implementations, client device 1106 may be equipped with one or more vision sensors having one or more fields of view, although this is not required. Vision sensor(s) 1107 may take various forms, such as digital cameras, passive infrared (“PIR”) sensors, stereoscopic cameras, RGBd cameras, etc. The one or more vision sensors 1107 may be used, e.g., by an image capture module 11121, to capture image frames (still images or video) of an environment in which client device 1106 is deployed, as well as allowing for teleconferencing.

Additionally or alternatively, in some implementations, client device 1106 may include one or more proximity sensors 1105. Proximity sensor(s) may take various forms, such as passive infrared (“PIR”) sensors, radio frequency identification (“RFID”), a component that receives a signal emitted from another nearby electronic component (e.g., Bluetooth signal from a nearby user's client device, high- or low-frequency sounds emitted from the devices, etc.), and so forth. Additionally or alternatively, vision sensors 1107 and/or a microphone 1109 may also be used as proximity sensors, e.g., by visual and/or audibly detecting that a user is proximate.

As described in more detail herein, the treatment management module 1120 may engage in human-to-computer dialog sessions with one or more users via user interface input and output devices of one or more client devices 1106. In some implementations, the system 1100 may engage in a human-to-computer dialog session with a user in response to user interface input provided by the user via one or more user interface input devices of one of the client devices 1106.

Each of client computing device 1106 and computing device(s) operating cloud-based automated components 1119 may include one or more memories for storage of data and software applications, one or more processors for accessing data and executing applications, and other components that facilitate communication over a network. The operations performed by client computing device 1106 and/or by automated modules 1120 may be distributed across multiple computer systems. The modules 1120 may be implemented as, for example, as computer programs running on one or more computers in one or more locations that are coupled to each other through a network.

As noted above, in various implementations, client computing device 1106 may operate an automated client 1108, or client portion of the system 1100. In various implementations, automated client 1108 may include a speech capture module 1110 and a biometrics sensor module 1111. In other implementations, one or more aspects of speech capture module 1110 and image/video module 11121 may be implemented separately from automated client 1108, e.g., by one or more cloud-based automated components 1119. For example, in FIG. 11, there is also a cloud-based visual module 11122.

In various implementations, speech capture module 1110, which may be implemented using any combination of hardware and software, may interface with hardware such as a microphone 1109 or other pressure sensor to capture an audio recording of a user's utterance(s). Various types of processing may be performed on this audio recording for various purposes. In some implementations, biometrics sensor module 1111, which may be implemented using any combination of hardware or software, may be configured to interface with camera 1107 to capture one or more image frames (e.g., digital photographs) that correspond to a field of view of the vision sensor 1107. Embodiments of the system 1100 may use one or more artificial intelligence (or machine learning) models that are trained to generate output.

Speech input from a provider or a client may be sent to cloud-based automated components 1119, which may include a cloud-based text-to-speech (“TTS”) module 1116 and/or a cloud-based STT module 1117. Cloud-based TTS module 1116 may be configured to leverage the virtually limitless resources of the cloud to convert textual data into computer-generated speech output. In some implementations, TTS module 1116 may provide the computer-generated speech output to client device 1106 to be output directly, e.g., using one or more speakers. In other implementations, textual data) may be provided to speech capture module 1110, which may then convert the textual data into computer-generated speech that is output locally. Cloud-based STT module 1117 may be configured to leverage the virtually limitless resources of the cloud to convert audio data captured by speech capture module 1110 into text, which may then be provided to a client or provider. In some implementations, cloud-based SIT module 1117 may convert an audio recording of speech to one or more phonemes, and then convert the one or more phonemes to text.

An interface module 1115 may store and retrieve Information from a database 1114 or other data repository. The database 1114 may be cloud-based or comprising part of the client device 1106 or other hardware connected to the client device 1106. The database 1114 may store or otherwise have access to data and algorithms 1114₁-1114ₙ. A location determination module 1113 may include a gyroscope and/or global positioning system (GPS) hardware and software, among other tracking systems.

The cloud-based automated components 1119 may include algorithms 1122, the aforementioned ITS module 1116, the aforementioned SIT module 1117, and other components that are described in more detail below. In some implementations, one or more of the modules and/or modules of the components 1119 may be omitted, combined, and/or implemented in a component that is separate from the components 1119. In some implementations, one or more of the components of, such as algorithms/program code 1122, TTS module 1116, STT module 1117, real-time tracking module 1124, etc., may be implemented at least on part on client devices 1106 (e.g., to the exclusion of the cloud). A treatment management module 1128 may access a database 1129.

In some implementations, the components 1119 may serve as an intermediary between users and one or more third party computing services 1130 (or “third party agents”, or “agents”). These third party computing services 1130 may be independent software processes that receive input and provide responsive output. Some third party computing services may take the form of third party applications that may or may not operate on computing systems that are separate from those that operate, for instance, cloud-based automated components 1119.

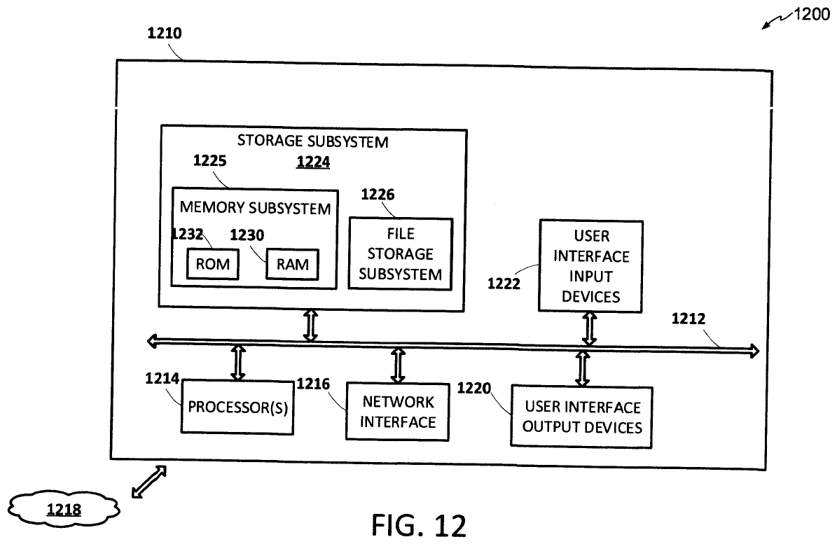

FIG. 12 is a block diagram of an example computing device 1210 that may optionally be utilized to perform one or more aspects of techniques described herein. In some implementations, one or more of a client computing device, user-controlled resources engine, and/or other component(s) may comprise one or more components of the example computing device 1210.

Computing device 1210 typically includes at least one processor 1214 that communicates with a number of peripheral devices via bus subsystem 1212. These peripheral devices may include a storage subsystem 1224, including, for example, a memory subsystem 1225 and a file storage subsystem 1226, user interface output devices 1220, user interface input devices 1222, and a network interface subsystem 1216. The user interface input devices 1222 of an implementation may include a response volume setting, among other features. The input and output devices allow user interaction with computing device 1210. Network interface subsystem 12:16 provides an interface to outside networks and is coupled to corresponding interface devices in other computing devices.

User interface input devices 1222 may include a keyboard, pointing devices such as a mouse, trackball, touchpad, or graphics tablet, a scanner, a touchscreen incorporated into the display, audio input devices such as voice recognition systems, microphones, and/or other types of input devices. In general, use of the term “input device” is intended to include all possible types of devices and ways to input information into computing device 1210 or onto a communication network.

User interface output devices 1220 may include a display subsystem, a printer, a fax machine, or non-visual displays such as audio output devices. The display subsystem may include a cathode ray tube (CRT), a flat-panel device such as a liquid crystal display (LCD), a projection device, or some other mechanism for creating a visible image. The display subsystem may also provide non-visual display such as via audio output devices. In general, use of the term “output device” is intended to include all possible types of devices and ways to output information from computing device 1210 to the user or to another machine or computing device.

Storage subsystem 1224 stores programming and data constructs that provide the functionality of some or all of the modules described herein. For example, the storage subsystem 1224 may include the logic to perform selected aspects of the method and to implement various components depicted in other figures herein.

These software modules are generally executed by processor 1214 alone or in combination with other processors. Memory 1225 used in the storage subsystem 1224 may include a number of memories including a main random access memory (RAM) 1230 for storage of instructions and data during program execution and a read only memory (ROM) 1232 in which fixed instructions are stored. A file storage subsystem 1226 can provide persistent storage for program and data files, and may include a hard disk drive, a floppy disk drive along with associated removable media, a CD-ROM drive, an optical drive, or removable media cartridges. The modules implementing the functionality of certain implementations may be stored by file storage subsystem 1226 in the storage subsystem 1224, or in other machines accessible by the processor(s) 1214.

Bus subsystem 1212 provides a mechanism for letting the various components and subsystems of computing device 1210 communicate with each other as intended. Although bus subsystem 1212 is down schematically as a single bus, alternative implementations of the bus subsystem may use multiple busses.

The computing device 1210 can be of varying types including a workstation, server, computing cluster, blade server, server farm, or any other data processing system or computing device. Due to the ever-changing nature of computers and networks, the description of computing device 1210 depicted in FIG. 12 is intended only as a specific example for purposes of illustrating some implementations. Many other configurations of computing device 1210 are possible having more or fewer components than the computing device depicted in FIG. 12.

While several implementations have been described and illustrated herein, a variety of other means and/or structures for performing the function and/or obtaining the results and/or one or more of the advantages described herein may be utilized, and each of such variations and/or modifications is deemed to be within the scope of the implementations described herein. More generally, all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific implementations described herein. It is, therefore, to be understood that the foregoing implementations are presented by way of example only and that, within the scope of the appended claims and equivalents thereto, implementations may be practiced otherwise than as specifically described and claimed. Implementations of the present disclosure are directed to each individual feat re, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

What is claimed is:

1. A method implemented by one or more processors of a device to treat an addiction, the method comprising:
providing a database of addiction treatment and patient data;
providing real-time data;
applying an algorithm to the real-time and databases data to generate a quantifiable value of a status of a patient, wherein the status relates to at least one of a withdrawal state or a detection of drug usage.

2. The method of claim 1, wherein generating the quantifiable value includes at least one of a daily withdrawal curve and a resiliency score.

3. The method of claim 1, further comprising alerting a user when a clinical threshold has been reached.

4. The method of claim 1, further comprising using biometrics to distinguish between physiological stress and psychological stress.

5. The method of claim 1, wherein generating the quantifiable value includes relapse risk stratification.

6. The method of claim 1, further comprising automatically integrating electronic health records from a clinic health information exchange data.

7. The method of claim 1, wherein generating the quantifiable value includes automatically linking to medical software.

8. The method of claim 1, wherein generating the quantifiable value includes automatically generating current procedural terminology (CPT) codes.

9. The method of claim 1, further comprising generating metrics to export for patient billing support.

10. The method of claim 1, further comprising using natural language processing for inputting patient data.

11. The method of claim 1, further comprising generating reports displaying the quantifiable value of the withdrawal status of the patient.

12. An apparatus comprising:
a processor;
a memory in communication with the processor, wherein the memory stores instructions that, in response to execution of the instructions by the processor, cause the processor to perform the following operations:
accessing stored addiction treatment and patient data;
receiving real-time data;
applying an algorithm to real-time and stored data to generate a quantifiable value of a status of a patient, wherein the status relates to at least one of a withdrawal state or a detection of drug usage.

13. The apparatus of claim 12, wherein generating the quantifiable value includes at least one of a daily withdrawal curve and a resiliency score.

14. The apparatus of claim 12, wherein the algorithm alerts a user when a clinical threshold has been reached.

15. The apparatus of claim 12, wherein the algorithm uses biometrics to distinguish between physiological stress and psychological stress.

16. The apparatus of claim 12, wherein generating the quantifiable value includes relapse risk stratification.

17. The apparatus of claim 12, wherein the algorithm integrates electronic health records from a clinic health information exchange data.

18. The apparatus of claim 12, wherein the algorithm automatically links to medical software.

19. The apparatus of claim 12, wherein generating the quantifiable value includes at least one of geographic data and mapping data.

20. At least one non-transitory computer-readable medium comprising instructions that, in response to execution of the instructions by one or more processors, cause the one or more processors to perform the following operations:
access stared addiction treatment and patient data;
receive real-time data;
apply an algorithm to real-time and stored data to generate a quantifiable value of a status of a patient, wherein the status relates to at least one of a withdrawal state or a detection of drug usage.
